(19) United States
(12) Patent Application Publication
(10) Pub. No.: US 2006/0263255 A1
(43) Pub. Date: Nov. 23, 2006

(54) Nanoelectronic sensor system and hydrogen-sensitive functionalization

(76) Inventors: Tzong-Ru Han, Cambridge, MA (US); Alexander Star, Pittsburgh, PA (US); Jean-Christophe P. Gabriel, Pinole, CA (US); Sergei Skarupo, San Francisco, CA (US); John Loren Passmore, Berkeley, CA (US); Philip G. Collins, Irvine, CA (US); Keith Bradley, New York, NY (US); Darin Olson, Menlo Park, CA (US)

Correspondence Address: O'MELVENY & MYERS LLP, 610 NEWPORT CENTER DRIVE, 17TH FLOOR, NEWPORT BEACH, CA, 92660, US

(21) Appl. No.: 11/354,561

(22) Filed: Feb. 14, 2006

Related U.S. Application Data
(63) Continuation-in-part of application No. 10/655,529, filed on Sep. 4, 2003, now abandoned.
(63) Continuation-in-part of application No. 10/945,803, filed on Sep. 20, 2004.
(63) Continuation-in-part of application No. 11/111,121, filed on Apr. 20, 2005.
(60) Provisional application No. 60/652,883, filed on Feb. 15, 2005.
(60) Provisional application No. 60/408,362, filed on Sep. 4, 2002.
(60) Provisional application No. 60/504,663, filed on Sep. 18, 2003.
(60) Provisional application No. 60/564,248, filed on Apr. 20, 2004.

(51) Int. Cl. G01N 33/00

(52) U.S. Cl. 422/83

(57) ABSTRACT

A new sensing technology for chemical/biomolecular sensors is provided. One such sensor detects molecular hydrogen (H₂) using nanoelectronic components. A tiny, low-cost nanosensor chip can offer: (i) performance that matches or exceeds that of existing technology, (ii) plug-and-play simplicity with both digital and analog control systems, and (ii) the small size and low power consumption needed for wireless integration.

### CROSS-REFERENCE TO RELATED APPLICATIONS

This application claims priority This application claims priority as a non-provisional application to U.S. Provisional Patent Application No. 60/652,883 filed Feb. 15, 2005, entitled “Nanoelectronic Sensor System And Hydrogen-Sensitive Functionalization”, which application is incorporated by this reference in its entirety for all purposes.

This application is also a continuation-in-part of and claims priority to U.S. patent application Ser. No. 10/655,529 filed Sep. 4, 2003 entitled “Improved Sensor Device With Heated Nanostructure”, which in turn claims priority to U.S. Provisional Patent Application No. 60/408,362 filed Sep. 4, 2002, which applications are incorporated by this reference in their entirety for all purposes.

This application is also a continuation-in-part of and claims priority to U.S. patent application Ser. No. 10/945,803 filed Sep. 20, 2004 entitled “Multiple Nanoparticles Electrodeposited On Nanostructures” (published as 2005-0157,445), which in turn claims priority to U.S. Provisional Patent Application No. 60/504,663 filed Sep. 18, 2003, which applications are incorporated by this reference in their entirety for all purposes.

This application is also a continuation-in-part of and claims priority to U.S. patent application Ser. No. 11/111,121 filed Apr. 20, 2005 entitled “Remotely Communicating, Battery-Powered Nanostructure Sensor Devices”, which in turn claims priority to U.S. Provisional Patent Application Ser. No. 60/564,248 filed Apr. 20, 2004, which applications are incorporated by this reference in their entirety for all purposes.

In addition, the following patent applications relate to the inventions herein in some manner and are incorporated by this reference in their entirety for all purposes: U.S. patent application Ser. No. 10/388,701 filed Mar. 14, 2003 entitled “Modification Of Selectivity For Sensing For Nanostructure Device Arrays” (published as US 2003-0175161); U.S. patent application Ser. No. 10/177,929 filed Jun. 21, 2002 entitled “Dispersed Growth Of Nanotubes On A Substrate” (corresponding to published WO04-040,671); U.S. patent application Ser. No. 10/280,265 filed Oct. 26, 2002 entitled “Sensitivity Control For Nanotube Sensors” (published as US 2004-0043527); and U.S. patent application Ser. No. 10/846,072 filed May 14, 2004 entitled “Flexible Nanotube Transistors” (published as US 2005-0184,641).

### BACKGROUND OF THE INVENTION

1. Field of the Invention

The present invention relates to nanostructure devices, such as nanotube sensors and transistors, in particular for the detection and measurement chemicals and biomolecules, such as gaseous hydrogen, and methods for fabricating the same.

2. Description of Related Art

A number of technical approaches have been developed for the measurement of chemical/biomolecular species, such as molecular hydrogen as a constituent of air and other analyte mediums. Examples include catalytic combustion sensors, infrared (IR) sensors, metal oxide semiconductor (MOS) sensors, gas chromatography, electrochemical sensors, surface acoustic wave (SAW) sensors, and the like.

With respect to hydrogen as an analyte, sensors and detection devices are made and distributed by various manufacturers, for example the DrägerSensor® H2 is distributed in the U.S. by Draeger Safety Inc. of Pittsburgh, Pa.; and the 3MHYT CiTiceL® is made and distributed by City Technology Ltd of Portsmouth, Hampshire, United Kingdom.

There has been interest in employ solid state electronic devices to function as chemical/biomolecular sensors, with a goal to employing the wafer-level mass production technology of the semiconductor industry to produce low-power, compact, inexpensive, electronically-readable sensors for a wide range of industrial applications.

One approach is to treat one or more elements of the device (e.g., a doped silicon substrate resistor, transistor, CMOS device and the like) so as to produce a sensitivity to a target analyte (functionalization), the sensitivity being intended to produce a measurable change in the electrical device characteristics in the presence of the analyte relative to its absence. In many application fields, the wide spread industrial use of such solid state chemosensors has been limited due to problems of limited sensitivity, high cross-sensitivity to non-target species, slow reaction and/or recovery times, limited concentration response range, device poisoning, elevated temperature requirements, limited service life, and the like.

However, there is a need for smaller, lower power, and lower cost chemical/biomolecular sensors, such as H₂ sensors, having a large dynamic range and fast response characteristics, and particularly sensors suitable to inclusion in multi-analyte arrays. The provision of a H2 sensor suitable for use in disposable units and/or suitable for integration in wireless self contained units can permit H2 sensors to be used much more widely than they are currently.

### SUMMARY OF THE INVENTION

Exemplary embodiments having aspects of the invention include a new sensing technology for chemical/biomolecular sensors, such as molecular hydrogen (H₂), that uses nanoelectronic components. A tiny, low-cost nanosensor chip can offer: (i) performance that matches or exceeds that of existing technology, (ii) plug-and-play simplicity with both digital and analog control systems, and (ii) the small size and low power consumption needed for wireless integration.

Exemplary embodiments having aspects of the invention include field effect transistors made from semiconducting single-walled carbon nanotubes (NTFETs) which provide a platform for sensitive chemical and biomolecule sensors, including H2 sensors.

Exemplary embodiments having aspects of the invention include integrated arrays having a plurality of sensor devices, such as one or more NTFET H2 sensors. The array may include sensors which provide a plurality of different functionalization materials, e.g., functionalizations targeted to different analytes, and/or functionalizations configured to provide differential response to a selected target analyte so as to enhance detection specificity, concentration range and/or dynamic range.

Exemplary embodiments having aspects of the invention include substrates having diffusion barrier layers disposed over the substrate base comprising a material substantially impermeable to the diffusion of metallic elements during carbon nanotube growth upon the substrate by chemical vapor deposition, such as Si3N4. Certain substrate embodiments also include a second layer disposed over the substrate and comprising a material configured to favor the catalytic growth of carbon nanotubes, such as a nano-smooth surface layer of SiO2. Alone or in combination, the diffusion barrier layer and/or the CNT-favoring surface layer may provide a gate dielectric, e.g. for a doped silicon substrate gate.

Exemplary embodiments of measurement methods and apparatus which reduce distortions in measurements of channel current in NTFET embodiments due to parasitic capacitance of source and drain contact pads adjacent a substrate including a back gate.

### BRIEF DESCRIPTION OF THE DRAWINGS

FIG. 1A is a schematic cross section view of a nanostructure device having aspects of the invention with a recognition layer specific to a selected analyte.

FIG. 1B is a cross-sectional diagram which illustrates an exemplary electronic sensing device, similar in a number of respects to the device of FIG. 1A, configured in this example as a capacitance sensor for detecting an analyte.

FIG. 1C is a diagram illustrating parasitic capacitance in measurements of source and drain currents in an NTFET.

FIG. 1D is a diagram illustrating a measurement apparatus and method for reducing distortions due to parasitic capacitance in measurements of source and drain currents in an NTFET.

FIG. 2 is a schematic cross section view of a substrate comprising a diffusion barrier having aspects of the invention to protect the substrate from catalyst migration.

FIGS. 3A and 3B are schematic cross section views of a portion of an example of a nanotube NFET device, both without (FIG. 3A) and including (FIG. 3B) a diffusion barrier having aspects of the invention, illustrating the containment of a contaminant (iron containing catalyst in this example).

FIGS. 4A and 4B are plots of conductance vs. gate voltage which illustrate the effect of palladium functionalization on an NTFET device having aspects of the invention, FIG. 4A corresponding to a device having a non-functionalized nanotube network channel, and FIG. 4B corresponding to a device including palladium.

FIG. 5 is a plot showing the conductance versus time response of an exemplary palladium functionalized NT sensor having aspects of the invention, during short exposures to 100% hydrogen gas in an air environment.

FIG. 6 is a plot showing the dependence on concentration of hydrogen of the response of an exemplary palladium functionalized NT sensor having aspects of the invention.

FIG. 7 is a plot showing the relative change in conductance vs. hydrogen concentration of an exemplary palladium functionalized NT sensor having aspects of the invention, in air.

FIG. 8 is a plot illustrating the cross-selectivity of an exemplary palladium functionalized NT sensor having aspects of the invention, showing the conductance versus time dependence during short exposures to hydrogen (H2), carbon monoxide (CO), and methane (CH4) gases.

FIG. 9 is a plan view of an exemplary NT sensor having aspects of the invention, showing an interdigitated layout of source and drain electrodes, patterned so as to be connected by an nanotube network.

FIG. 10 is a circuit diagram illustrating the measurement system employed in the embodiment having aspects of the invention as described in Example B.

FIGS. 11 and 12 are photographs showing an exemplary wireless remote H2 sensor system (FIG. 9) and a corresponding exemplary base receiver system for the sensor, coupled so as to activate a conventional alarm system (FIG. 10), each having aspects of the invention.

FIG. 13 is a plot showing the response and recovery of an exemplary wireless remote H2 sensor system such as shown in FIGS. 9 and 10 to exposure to a brief pulse of hydrogen at a concentration of about 20,000 ppm in air.

FIG. 14 is a schematic cross section view of a nanostructure device having aspects of the invention including passivation and encapsulation of device elements.

### DETAILED DESCRIPTION OF THE EMBODIMENTS

It should be understood that one aspect of the invention herein may be set forth in one part of the description, figures, formulas, and/or examples herein, while other aspects of the invention may be set forth in other parts of the description, figures, formulas, and/or examples herein. Certain advantageous inventive combinations may be taught in one part of the description, figures, formulas, and/or examples herein, and the detailed description and best mode of such combinations and their respective elements may be set forth in other parts of the description, figures, formulas, and/or examples herein. The specification is to be understood as read in its entirety. Therefore the invention is to be defined by the claims herein.

### 1. NANOSENSOR ARCHITECTURE

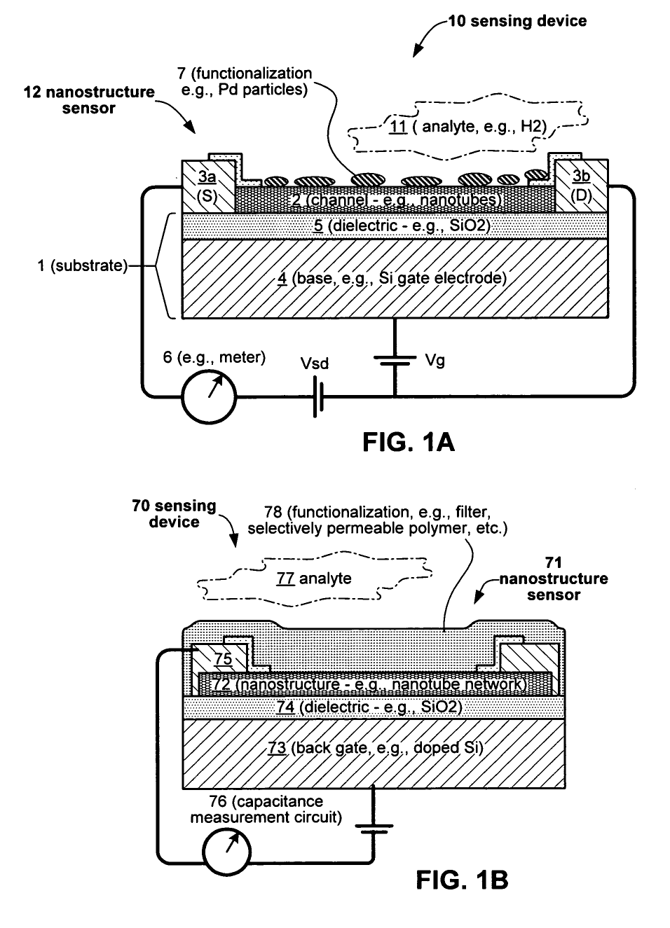

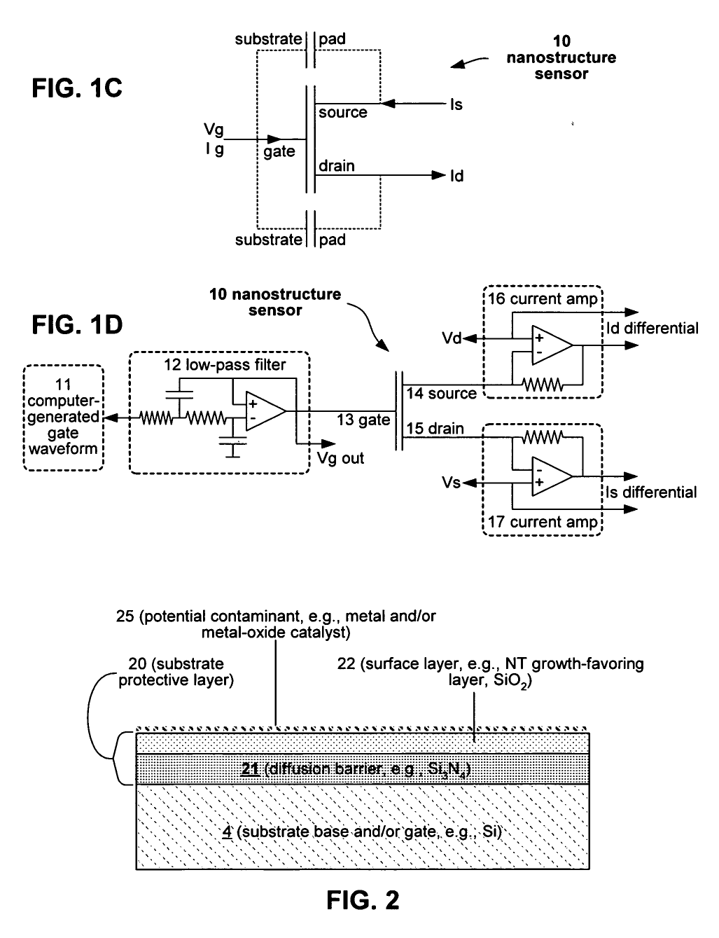

FIG. 1. shows an electronic sensing device 10 for detecting an analyte 11 (e.g. hydrogen gas), comprising a nanostructure sensor 12. Device 12 comprises a substrate 1, and a conducting channel or layer 2 including nanostructured material disposed upon a substrate 1.

The nanostructured material may contact the substrate as shown, or in the alternative, may be spaced a distance away from the substrate, with or without a layer of intervening material. In an embodiment of the invention, conducting channel 2 may comprise one or more carbon nanotubes. For example, conducting channel 2 may comprise a plurality of nanotubes forming a mesh, film or network. In preferred embodiments the carbon nanotubes (CNT) comprise single-walled carbon nanotubes (SWNTs).

At least two conductive elements or contacts 3a, 3b may be disposed over the substrate and electrically connected to conducting channel 2. Elements 3a, 3b may comprise metal electrodes in direct contact with conducting channel 2. In the alternative, a conductive or semi-conducting material (not shown) may be interposed between contacts 3a, 3b and conducting channel 2. contacts 3a, 3b may comprise a source electrode S and a drain electrode D upon application of a selected and/or controllable source-drain voltage Vsd (note that the voltage and/or polarity of source relative to drain may be variable, e.g., current may be DC, AC and/or pulsed, and the like).

It is often advantageous to operate the device 10 as a gate-controlled field effect transistor, and sensor 12 may further comprise a gate electrode 4. In certain embodiments, the gate 4 comprises a base portion of the substrate, such as doped-silicon wafer material isolated from contacts 3a, 3b and channel 2 by dielectric 5, so as to permit a capacitance to be created by an applied gate voltage Vg. For example, the substrate 1 may comprise a silicon back gate 4, isolated by a dielectric layer 5 of SiO2. Such embodiments may be referred to as an NTFET (nanotube field effect transistor)

In certain embodiments, sensor 12 may further comprise a layer of inhibiting or passivation material 6 covering regions adjacent to the connections between the conductive elements 3a, 3b and conducting channel 2. The inhibiting material may be impermeable to at least one chemical species, such as the analyte 11. The inhibiting material may comprise a passivation material as known in the art, such as silicon dioxide, aluminum oxide, silicon nitride, and the like. Further details concerning the use of inhibiting materials in a NTFET are described in prior application Ser. No. 10/280,265, filed Oct. 26, 2002, entitled “Sensitivity Control For Nanotube Sensors” (published as US 2004-0043527 on Mar. 4, 2004) which is incorporated by reference herein.

The conducting channel 2 (e.g., a carbon nanotube layer) is typically functionalized to produce a sensitivity to one or more target analytes 11. Although nanoparticles such as carbon nanotubes may respond to a target analyte through charge transfer or other interaction between the device and the analyte, more generally a specific sensitivity can be achieved by employing recognition material 7 that induces a measurable change the device characteristics upon interaction with a target analyte. Typically, the sensor functionalization layer 7 is selected for a specific application. The analyte may produce the measurable change by electron transfer, and/or may influence local environment properties, such as pH and the like, so as to indirectly change device characteristics. Alternatively or additionally, the recognition material may induce electrically-measurable mechanical stresses or shape changes in the conducting channel 2 upon interaction with a target analyte.

In a typical embodiment having aspects of the invention, the sensitivity is produced and/or regulated by the association of the nanotube layer 2 with a functionalization material, e.g. disposed as a functionalization layer 7 adjacent channel 2. The functionalization layer 7 may be of a composition selected to provide a desired sensitivity to one or more target species or analytes. The functionalization material may be disposed on one or more discrete portions of the device, such as on all or a portion of the channel 2, or alternatively may be dispersed over the sensor 12, such as on contacts 3 and/or exposed substrate 1.

Device 10 preferably includes suitable circuitry in communication with sensor elements to perform electrical measurements. For example, a conventional power source may supply a source drain voltage Vsd between contacts 3a, 3b. Measurements via the sensor device 10 may be carried out by circuitry represented schematically by meter 6 connected between contacts 3a, 3b. In embodiments including a gate electrode 4, a conventional power source may be connected to provide a selected and/or controllable gate voltage Vg. Device 10 may include one or more electrical supplies and/or a signal control and processing unit (not shown) as known in the art, in communication with the sensor 12.

Optionally device 10 may comprise a plurality of sensors 12 disposed in a pattern or array, as described in U.S. patent application Ser. No. 10/388,701 filed Mar. 14, 2003 entitled “Modification Of Selectivity For Sensing For Nanostructure Device Arrays” (now published as US 2003-0175161). Each device in the array can be functionalized with identical or different functionalization. Identical device in an array can be useful in order to multiplex the measurement to improve the signal/noise ratio or increase the robustness of the device by making redundancy.

### 2. SENSOR ELEMENTS

Substrate. The substrate 1 may be insulating, or alternatively the substrate 1 may comprise a layered structure, having a base 4 and a separate dielectric layer 5 disposed to isolate the contacts 3 and channel 2 from the substrate base 4. The substrate (1) may comprise a rigid or flexible material, which may be conducting, semiconducting or dielectric, and the like, and may which may include a multilayer and/or composite structure having constituents of different properties and compositions. Suitable substrate materials include quartz, alumina, polycrystalline silicon, III-V semiconductor compounds, and the like.

Substrate materials may be selected to have particular useful properties, such as transparency, microporosity, magnetic properties, monocrystalline, polycrystalline and/or amorphous, and the like, or combinations thereof. For example, in certain embodiments having aspects of the invention, the substrate 1 includes a silicon wafer doped so as to function as a back gate electrode 4, the wafer being coated with intermediate diffusion barrier of Si3N4 and an upper dielectric layer of SiO2. Optionally, additional electronic elements may be integrated into the substrate for various purposes, such as thermistors, heating elements, integrated circuit elements and the like.

In certain alternative embodiments, the substrate may comprise a flexible insulating polymer, optionally having an underlying gate conductor (such as a flexible conductive polymer composition), as described in U.S. patent application Ser. No. 10/846,072 filed May 14, 2004 entitled “Flexible Nanotube Transistors”, the entirety of which application is incorporated herein by this reference.

In further alternative embodiments, the substrate may comprise a microporous material permitting suction to be applied across the substrate, e.g., porous alumina for vacuum deposition of a nanotube network channel 2 from suspension or solution, as described in U.S. Patent Application No. 60/639954, filed December 28, 2004, entitled “Nanotube Network-On-Top Architecture For Biosensor”, the entirety of which application is incorporated herein by this reference, including the incorporation of the therein cited publications.

Contacts. The conductor or contact (3) used for the source and drain electrodes can be any of the conventional metals used in semiconductor industry, or may be selected from Au, Pd, Pt, Cr, Ni, ITO, W or other metallic material or alloy or mixture thereof. Alternatively, the contact may comprise a multi-layer or composite of metals/metallic materials, such as Ti+Au, Cr+Au, Ti+Pd, Cr+Pd, or the like. Such multiplayer can help in improving the adhesion of the metal to the substrate. Alternatively other conductive materials may be employed, such as conductive polymers and the like. In one exemplary embodiment, electrical leads were patterned on top of a nanotube network channel from titanium films 30 nm thick capped with a gold layer 120 nm thick.

The dimension of the gap between source and drain may be selected to suit or optimize characteristics for a particular application. It should be understood that one or more of each of a source and drain electrode may be arranged in an interdigitated and/or spaced-apart electrode array, permitting a comparative large area of nanostructured layer 2 having a comparatively small source-drain gap to be arranged compactly.

Although gate electrode 4 may comprise materials generally similar to contacts 3, often the gate electrode 4 comprises a sublayer within substrate 1. Alternatively, the gate electrode 4 may comprise doped silicon, patterned metal, ITO, other conductive metal or non-metal material, and the like, or combinations thereof. Alternative forms of gate electrodes may be employed (not shown), such as a top gate, a gate effected via a conducting analyte carrier medium (e.g. an aqueous solution), and the like. Optionally, a device 12 may comprise such other electrodes as a counter electrode, a reference electrode, a pseudo-reference electrode, and the like without departing from the spirit of the invention.

Conducting Channel Or Nanostructured Layer. Exemplary embodiments having aspects of the invention include sensor devices having at least one conducting channel 2 comprising one or more of a number of types of nanostructure particles without departing from the spirit of the invention. For example, conducting channel or layer 2 may comprises one or more single-wall carbon nanotubes, multiple-wall carbon nanotubes, nanowires, nanofibers, and/or nanorods, nanospheres, and the like. In addition, or in the alternative, conducting channel or layer 2 may comprise boron, boron nitride, and carbon boron nitride, silicon, germanium, gallium nitride, zinc oxide, indium phosphide, molybdenum disulphide, silver, or other suitable material.

Conducting Channel Comprising Nanoparticle Network. In certain embodiments, conducting channel or nanostructure layer 2 comprises an interconnected network of smaller nanoparticles disposed to form a percolation layer, mesh, or film which provides in operation at least one electrical conduction path between a source electrode S and a drain electrode D. In such a network of nanoparticles, it is not necessary that any single nanoparticle extends entirely between the source and drain contacts 3a, 3b, and in operation the conductivity of channel 2 between source electrode S and drain electrode D may be maintained by interconnections, contacts or communications between adjacent nanoparticles. Such networks of nanoparticles, such as nanotubes and the like, may be configured to be defect-tolerant, in that disruption of any particular conductive path may be compensated by remaining paths within the network.

In certain preferred embodiments having aspects of the invention, the nanostructured conducting channel 2 includes one or more single walled and/or multi walled carbon nanotubes, which may be arranged as clumps or bundles, and/or as distinct separated fibers. A useful network of nanotubes may be provided, for example, by distributing a dispersion of nanotubes over a substrate so as to be approximately planar and randomly oriented. For example, in certain preferred embodiments having aspects of the invention, conducting channel 2 comprises a network including a plurality of dispersed single wall carbon nanotubes (SWCNT) in which the nanotubes are oriented substantially randomly, non-parallel and unclumped with respect to one another as an interconnecting mesh disposed generally parallel to the substrate.

Note that the electrical characteristics of the channel may be optimized to suit a particular functionalization chemistry or other constituent of the sensor which effects conductivity, or to suit a desired range of analyte concentration. In preferred embodiments, the density and/or thickness of a nanotube network, and/or the composition of the nanotubes regarding metallic versus semiconducting properties may be varied to provide a desired degree of conductivity between the source and drain pairs(s) (S and D).

One advantage of a network architecture for the conducting channel 2 is that these factors may be varied to produce a conducting network having a selected margin above (or below) the percolation limit, permitting convenient optimization of device characteristics. For example, a NT network channel may be formed to be slightly below the percolation limit for the uncoated network, and modified by deposition of a conducting recognition material, such as Pd, to result in a functionalized channel of desired conductivity. In another example, the conductivity of an initially dry network may be selected to allow for operation in association with anticipated additional conductivity of a fluid analyte medium, such as a physiologic buffer or the like.

In addition, a conducting channel 2 comprising a generally random dispersion of individual nanoparticles is that a “statistical,” rather than a “localized” approach to nanostructure device fabrication can be used, permitting cost savings and mass production techniques. In the “statistical” approach, electrical contacts can be placed anywhere on the dispersion of individual nanostructures to form devices, without a specific correspondence between electrode position and any particular nanoparticle position, the dispersion of nanoparticles ensuring that any two or more electrodes placed thereon can form a complete electrical circuit with functioning nanostructures providing the connection. By distributing a large plurality of randomly oriented nanotubes in a dispersion over (or under) an electrode array, uniform electrical properties in the individual devices can be assured with higher yields and faster processing than is possible using the prior art approach of controlled placement or growth of nanotubes.

Nanoparticle Network Formation. Suitable nanoparticle networks may be formed by alternative methods. In certain embodiments having aspects of the invention the conducting layer 2 comprises a an interconnecting network of single-wall carbon nanotubes formed directly upon the substrate, e.g. by reactions of vapors in the presence of a catalyst or growth promoter disposed upon the substrate. For example single-walled nanotube networks can be grown on silicon or other substrates by chemical vapor deposition from iron-containing catalyst nanoparticles with methane/hydrogen gas mixture at about 900 degree C. Other catalyst materials and gas mixtures can be used to grow nanotubes on substrates, and other electrode materials and nanostructure configurations and are described by Gabriel et al. in U.S. patent application Ser. No. 10/099,664 filed Mar. 15, 2002 entitled “Modification Of Selectivity For Sensing For Nanostructure Sensing Device Arrays”, and in International Application PCT/US03/19,808 filed Jun. 20, 2003, entitled “Dispersed Growth Of Nanotubes On A Substrate” and published as WO2004-040,671 on May 13, 2004, both of which applications are incorporated by reference herein.

Advantageously, the use of highly dispersed catalyst or growth-promoter nanoparticles permits a network of nanotubes of controlled diameter and wall structure to be formed in a substantially random and unclumped orientation with respect to one another, distributed substantially evenly at a selected mean density over a selected portion of the substrate. The particle size distribution may be selected to promote the growth of particular nanotube characteristics (e.g., diameter, single wall versus multiple wall, and the like).

In certain embodiments having aspects of the invention the conducting layer (2) comprises an interconnecting network of nanoparticles laid down by deposition from a solution or suspension of nanostructures, such as a solution of dispersed carbon nanotubes. See for example, the methods described in the above incorporated U.S. patent application Ser. No. 10/846,072 filed May 14, 2004 entitled “Flexible Nanotube Transistors”. Such methods as spin coating, spray deposition, dip coating and ink-jet printing may be employed.

Alternatively, a sensor device embodiment having aspects of the invention, such as a functionalized NTFET sensor, may include a channel 2 comprising a nanotube network formed by suction deposition on a porous substrate or membrane by the methods described in U.S. Patent Application No. 60/639954, filed Dec. 28, 2004, entitled “Nanotube Network-On-Top Architecture For Biosensor”, which application is incorporated by reference. The network thus formed may be included either attached to its deposition membrane, or included separated form the deposition membrane, such as by membrane dissolution, transfer bonding, and the like. Methods of forming conductive layers of controlled density from nanotubes employing vacuum suction through a nano-scale porous membrane are described in L. Hu, D. S. Hecht, and G. Grüner, Percolation In Transparent And Conducting Carbon Nanotube Networks, Nano Lett. 2004, Vol. 4, No. 12, 2513-2517, which publication is incorporated here by reference as if fully set forth herein.

It should be understood that carbon nanotubes may exhibit either metallic or semiconductor properties, depending on the particular graphitic lattice orientation. Various methods may be employed to select a desired composition of nanotubes for the nanotube layer 2 of an operational device. In certain method embodiment, a plurality of generally similar nanotube devices are fabricated, such as an array of device dies disposed on a silicon wafer, wherein the plurality of devices have a range of electrical characteristics, e.g., due to differing metallic or semiconductor composition of nanotube layer 2. The fabricated dies may be individually tested, e.g. by automated or semi-automated pin probe test rigs. Dies exhibiting a selected electrical behavior or range of behavior may be marked and selected for further processing and use; and any non-conforming dies may be culled, or otherwise processed.

Alternatively Nanotube layer 2 may be constructed from preprocessed source nanotube material which includes a selected composition of metallic versus semiconductor properties (e.g., solely semiconductor nanotubes). Alternatively, the nanotube layer may be formed of an arbitrary mixture of nanotube composition, and the layer subsequently treated to selectively remove, oxidize, disconnect or deactivate all or a portion of the metallic nanotubes, e.g. by ohmic heating, so as to leave a conducting channel of selected properties (e.g., solely semiconductor nanotubes). The latter approach may be employed advantageously where the nanotube layer 2 is formed directly upon the substrate 1, for example by catalyst initiated CVD.

Functionalization or Recognition Layer. Functionalization 7 may be selected for a wide range of alternative chemical or biomolecular analytes. Examples include functionalization specific to gas analytes of industrial or medical importance, such as carbon dioxide. See U.S. patent application Ser. No. 10/940,324 filed Sep. 13, 2004 entitled “Carbon Dioxide Nanoelectronic Sensor”, which is incorporated herein by reference. See also U.S. patent application Ser. No. 10/656,898 filed Sep. 5, 2003 entitled “Polymer Recognition Layers For Nanostructure Sensor Devices”, which is incorporated herein by reference.

Examples include functionalization 7 specific to biomolecules, organisms, cell surface groups, biochemical species, and the like. See U.S. patent application Ser. No. 10/345,783 filed Jan. 16, 2003, entitled “Electronic Sensing Of Biological And Chemical Agents Using Functionalized Nanostructures” (now published as US 2003-0134433), and U.S. patent application Ser. No. 10/704,066 filed Nov. 7, 2003 entitled “Nanotube-Based Electronic Detection Of Biomolecules” (now published as US 2004-0132070), both of which applications are incorporated herein by reference.

Functionalization material 7 may be a single component or point reaction, a mixture or multilayer assembly, or a complex species (e.g., including both synthetic components and naturally occurring biomaterials). See, for example, U.S. application Ser. No. 10/388,701 filed Mar. 14, 2003 entitled “Modification Of Selectivity For Sensing For Nanostructure Device Arrays” (published as US 2003-0175161), and U.S. Application No. 60/604,293, filed Nov. 19, 2004, entitled “Nanotube Sensor Devices For DNA Detection”, which applications are incorporated herein by reference.

Functionalization material 7 (and other sensor elements) may be selected to suit various physical forms of sample media, such as gaseous and/or liquid analyte media. See, for example, U.S. patent application Ser. No. 10/773,631 filed Feb. 6, 2004 entitled “Analyte Detection In Liquids With Carbon Nanotube Field Effect Transmission Devices”, and U.S. Patent Application No. 60/604,293, filed Nov. 13, 2004, entitled “Nanotube Based Glucose Sensing”, both of which applications are incorporated herein by reference.

Functionalization of an H2 Sensor Embodiment. In an exemplary embodiment of an H₂ sensor having aspects of the invention, the functionalization layer 7 may comprise metal particles and/or larger aggregates For example, the composition of the functionalization material 7 may be a metal which has a catalytic or other interaction with hydrogen, such as Pd, Pt, Au, Ni, Ti, or alloys and/or combinations thereof. A preferred composition includes Pd, either alone or as an alloy, such as Pd/Ni 90/10% w/w or Pd/Ti 90/10 % w/w, and the like. Alternative alloys may be employed, such as Pd/Au, Pd/V, Pd/Cu, and the like. One particularly preferred alloy comprises Pd and about 16.3% w/w of Ni. Such materials can be evaporated on the sensor 12.

In addition, or in the alternative, metals may be electrodeposited specifically on the channel 2 (e.g., carbon nanotubes) as described in more detail in U.S. patent application Ser. No. 10/945,803 filed Sep. 20, 2004 entitled “Multiple Nanoparticles Electrodeposited On Nanostructures”, which is incorporated by reference herein, and in the above referenced U.S. application Ser. No. 10/388,701. The described methods of electrodeposition from solution (e.g. PdCl2) permit control of the particle size and particle distribution density within selected limits, and also permit selected target structures on a device to be coated with metal particles, while avoiding deposition on adjacent structures.

In certain embodiments of sensors having aspects of the invention, the functionalization material may be distributed more or less uniformly over the device surface. Alternatively, there may be advantages to a selective distribution of functionalization material. For example, in an exemplary embodiment of a hydrogen senor having aspects of the invention, a functionalization metal (e.g. Pd or a Pd alloy) may be deposited so as to only partially covers the nanotube layer and so that the functionalization metal is not substantially in contact with either the source or drain electrodes (S or D in FIGS. 1 and 4). For example, in certain embodiments, Pd or a Pd alloy may deposited by evaporation so that a plurality of small fields are created, called “bars”.

Note that the patterning of deposition of materials may employ photolythographic and masking technology such as used industrially for the making of integrated circuits. Alternatively, for appropriate feature sizes, shadow making, ink-jet printing other patterning techniques may be employed for selective application of functionalization and other materials. For example, sensor-specific functionalization, passivation or encapsulation materials may be applied in a plurality of different patterns to selected portions of an array of sensors, so as to produce a multifunctional sensor array.

By permitting some of the nanostructures not to be fully covered with metal, the electrical field created at the gate electrode may be modulated in a selected fashion by the functionalization metal. This may permit more functionalization metal to be incorporated in a device of a given size without an undesirable degree of screening, thereby enhancing the device response to the analyte (e.g. hydrogen). In addition using more functionalization metal may extend the useful lifetime of the device, particularly where oxidative or other decay of the functionalization metal is a life-limiting factor.

An additional advantage of the use of selective functionalization metal deposition (such as “Pd bars”) is to increase the dynamic range of the sensor device or sensor array system. In one exemplary embodiment the concentration of a relatively thick layer of functionalization metal in the bar gives a broader H2 concentration response range than a thin continuous functionalization metal coating which reaches saturation over a narrow range. In another example, an array of generally similar sensor devices, but having a range of sizes and/or number of bars, may be employed, with different sensors responding most sensitively to a different range of H2 concentration.

In certain alternative embodiments of a nanoelectronic sensor having aspects of the invention, a layer of passivation material may be deposited to separate functionalization or analyte recognition material from the channel, such as a nanotube network. For example, a layer of SiO2 may be deposited covering all or a portion of the nanotube network, followed by the deposition of Pd functionalization on top of the SiO2 layer. The dielectric layer may permit greater response of the sensor to variation in gate electrode voltage (e.g., a substrate gate electrode). Alternatively, conductive functionalization material may serve as a gate electrode, e.g. a layer of Pd functionalization metal or alloy separated from an NT network channel by a dielectric layer may be connected to an electrical lead, so as to permit a gate voltage to be applied to the functionalization material.

Sensor Arrays: A plurality of such sensor devices having aspects of the invention may be conveniently arranged as an array embodiment, the array being configured to provide for a number of advantageous measurement alternatives, as described in the patent applications incorporated by reference above. Note that there are a number of different measurement methods having aspects of the invention which are enabled by an array of sensors:
a) multiple analytes detected by a plurality of specifically functionalized sensors,
b) increased precision and dynamic range by a plurality of sensors each of which is optimized for a different range,
c) increased analyte specificity and flexibility by detecting a characteristic “profile” of responses of a selected target analyte to a plurality of differently-functionalized sensors,
d) self calibration systems and isolated reference sensors,
e) multiple use array having a plurality of addressable one-time-use sensor sub-units,
f) and the like, and combinations of these.

Alternative measurement systems. The electronic circuitry described in this example is by way of illustration, and a wide range of alternative measurement circuitry designs may be employed without departing from the spirit of the invention.

Embodiments of an electronic sensor device having aspects of the invention may include an electrical circuit configured to measure one or more properties of the nanotube sensor, such as measuring an electrical property via the conducting elements. Any suitable electrical property may provide the basis for sensor sensitivity, for example, electrical resistance, electrical conductance, current, voltage, capacitance, transistor on current, transistor off current, and/or transistor threshold voltage. Alternatively, sensitivity may be based on measurements including a combination, relationship, pattern and/or ratios of properties and/or the variation of one or more properties over time.

For example, a transistor sensor may be controllably scanned through a selected range of gate voltages, the voltages compared to corresponding measured sensor current flow (generally referred to herein as an I-Vg curve or scan). Such an I-Vg scan may be through any selected gate voltage range and at one or more selected source-drain potentials. The Vg range is typically selected from at least device “on” voltage through at least the device “off” voltage. The scan can be either with increasing Vg, decreasing Vg, or both, and may be cycled +− at any selected frequency.

From such measurements, and from derived properties such as hysteresis, time constants, phase shifts, and/or scan rate/frequency dependence, and the like, correlations may be determined with target detection and/or concentration and the like. The electronic sensor device may include and/or be coupled with a suitable microprocessor or other computer device of known design, which may be suitably programmed to carry out the measurement methods and analyze the resultant signals. Those skilled in the art will appreciate that other electrical and/or magnetic properties, and the like may also be measured as a basis for sensitivity. Accordingly, this list is not meant to be restrictive of the types of device properties that can be measured.

Capacitance or impedance measurements may be made response to an analyte, either solely or in combination with a transconductance measurement. For example, as shown in FIG. 1B, the capacitance of a CNT channel relative to a gate electrode may be measured (before, during and/or after analyte exposure), and may also be compared with contemporaneous measurements of channel resistance, conductance or other electrical properties. FIG. 1B is a cross-sectional diagram which illustrates an exemplary electronic sensing device 70 having aspects of the invention, similar in a number of respects to the device of FIG. 1A, configured in this example as a capacitance sensor for detecting an analyte, as further described in commonly invented and assigned U.S. Provisional Application No. 60/730,905 filed Oct. 27, 2005, entitled “Nanoelectronic Sensors And Analyzer System For Monitoring Anesthesia Agents And Carbon Dioxide In Breath”, which is incorporated by reference.

As shown in FIG. 1B, Sensor device 70 includes a nanostructure sensor 71 which includes a nanostructure conductive element 72, in this example a carbon nanotube network, disposed upon a substrate comprising a dielectric isolation layer 74 disposed upon a base 73, in this example a doped silicon wafer back gate. The nanotube network 72 is contacted by at least one conductive electrode 75 (a pair are shown, in this case having optional passivation on the electrode-nanotube contact region). The sensor device 70 further includes at least a capacitance measurement circuit 76 in electrical communication with contact 75 and back gate 73, so as to permit the capacitance and/or impedance of the spaced apart nanotube network/back gate assembly to be readily measured (i.e., the total charge required to be placed on either conductor to create a given voltage potential between conductors, C=Q/V). It should be understood that other capacitor conductors may be substituted for back gate 73 without departing from the spirit of the invention, such as a top gate, liquid gate, a second spaced-apart nanotube network conductor, and the like. Additionally, many alternative functional arrangements of the respective conductors are possible. The capacitance C of the sensor 71 may be calibrated, and compared analytically with the capacitance during exposure to analyte of interest 11. In particular, species having significant dipole moments may act to change the capacitance upon interaction with the nanotube network 72.

Compensation or avoidance of parasitic capacitance effects. An NTFET having aspects of the invention (such as a back-gated NTFET) may advantageously be employed with a variable “sweep” in gate bias voltage. However, there may exist significant parasitic capacitance effects, even a low sweep rates, due to the presence and relative properties of conductive electrode(s) or contact pads which electrically connect to the CNT channel material. The substrate gate has a capacitance with respect to a source or drain contact pad just as it has a capacitance with respect to the CNT channel. FIG. 1C is a diagram of this effect. Parasitic capacitance can lead to distorted I-Vg measurements. A variable gate voltage, such as a computer-generated stepped waveform, can cause further distortions due to aliasing effects.

FIG. 1D is a diagram of an exemplary measurement scheme embodiment with reduces distortions due to parasitic capacitance of an NTFET 10. A computer-generated signal 11 (which may have a fixed high update frequency) is passed through an analog lowpass filter 12 and applied to the gate electrode 13 of the NTFET 10. Synchronous measurements are taken at the gate voltage output and the source 14 and drain 15 currents (shown as outputs from current amps 16 and 17). Channel current may be computed as the differential of source and drain currents or (Id−Is)/2.

### 3. SUBSTRATE PROTECTIVE STRUCTURES

In certain embodiments of sensors having aspects of the invention, a substrate is employed which is sensitive to contamination during fabrication steps or other environmental exposure. For example, a semiconductor substrate such as a silicon wafer may be used, and it is desired that the electrical properties of the base portion of the wafer and/or dielectric coating be carefully controlled during processing to achieve desired insulating, semiconducting or conducting properties in particular portions of the completed sensor structure.

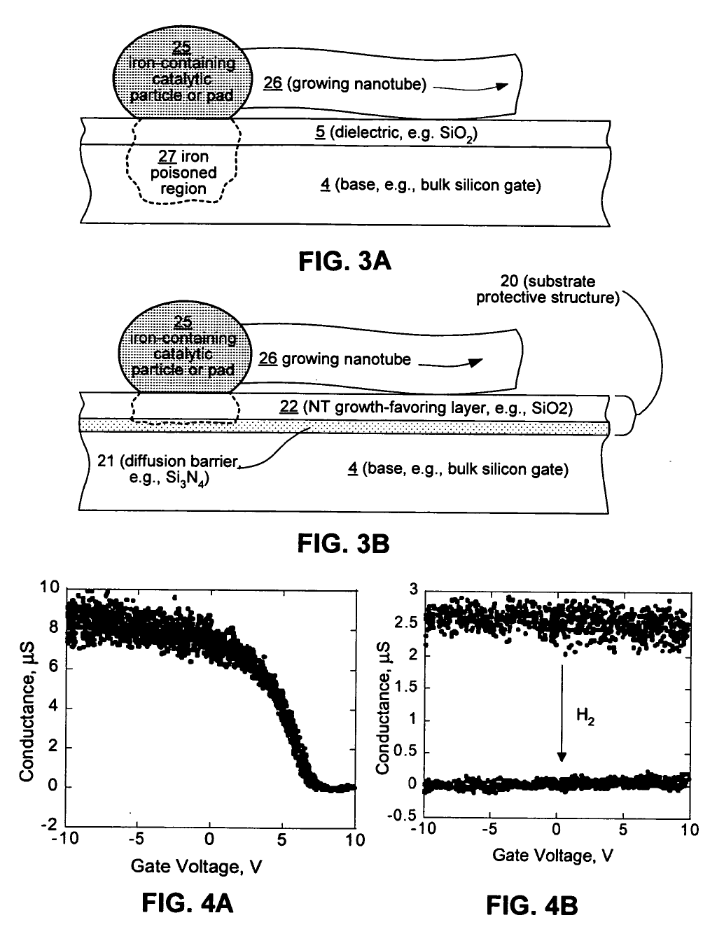

As shown schematically in cross section in FIGS. 2 and 3B, in one aspect of the invention a method and subassembly provides a novel substrate protective structure or layer 20 which includes diffusion barrier 21 protecting the substrate, The diffusion barrier 21, among other things, has the properties of protecting the underlying substrate material during fabrication and preventing contamination. In one example, metal catalysts or growth promoter 25 (e.g., iron or iron oxide) are employed to initiate CVD growth of nanotubes 26 (see the above incorporated International Application PCT/US03/19,808). When growing nanoparticles by chemical vapor deposition, a combination of high temperatures and reducing atmosphere are commonly used. As a consequence of these conditions, element migration or diffusion of metals is greatly increased.

Contaminant Migration. As shown in FIG. 3A, at the elevated CVD processing temperatures, iron can migrate from catalyst particle 25 through the dielectric 5 (e.g. SiO2) to contaminate the underlying substrate 4 in a “poisoned region” 27, so as to degrade the characteristics of the silicon substrate and/or dielectric layer. Such contamination may create a shorting conduction path from channel or contacts through the dielectric 5 to gate electrode 4, so as to bleed off the capacitance of the gate electrode, divert source-drain current, and/or degrade transistor on/off ratio.

It is possible to limit the migration into the substrate by increasing the thickness of the layer of SiO2 so that metals do not have the time to migrate through the whole layer during the exposure to elevated temperatures. However, this can however lead to very thick layers (more that 500 nm), and for efficient gate operation, it is typically desired to keep the thickness of the dielectric layer above the gate electrode as low as possible (to keep functional gate voltages as low as possible).

Diffusion Barrier. As shown in FIG. 3B, a diffusion barrier or contaminant protective layer having aspects of the invention may be included to isolate the NT promoting or catalyst material from the substrate material. Certain embodiments of a diffusion barrier having aspects of the invention employ a layer of Si3N4 to limit diffusion of metal growth promoters or other contaminants into the silicon substrate. It has been found that Si3N4 is more resistant to the diffusion of growth promoter elements than SiO2. In addition, the larger dielectric constant of Si3N4 also favors low gate voltages.

In one embodiment, a contaminant-protective layer comprises a layer of Si3N4, which is preferably from about 50 to about 3000 nm thick, and more preferably from about 50 to about 1000 nm thick (which may be formed by methods such as CVD, EPCVD, and the like).

Conditioned Surface Layer. In addition, the diffusion barrier, the substrate protective structure 20 may comprise a conditioned surface layer 22 overlying the diffusion barrier 21. In a number of alternative embodiments, specific surface characteristics, chemical properties or morphology is desired for the substrate. For example, it may be desired to have a conditioned surface layer with characteristics selected to promote attachment or uniform dispersal of a functionalization material.

In another example, embodiments of sensors in which nanotubes 26 or other nanoparticles are grown upon the substrate, certain surface characteristics are desired to favor nanoparticle growth, such as to obtain a desired density or length of nanotubes. A diffusion barrier such as a Si3N4 layer may be used as a surface layer alone to protect the bulk silicon substrate. However, it has also been found that an Si3N4 surface layer is less favorable to nanotube growth than a surface layer of SiO2, and it has been demonstrated that on surface Si3N4, fewer and shorter nanotubes are often grown versus a SiO2 surface.

The surface layer 22 may be configured to promote desired nanotube growth. In particular, SiO2 may be readily formed as a layer having a smooth surface texture at nanometer scales, so as to provide a suitable surface for the growth of a nanotube network. In a preferred example, a layer 22 of SiO2 is vapor deposited which has smooth nano-scale surface morphology. It has been demonstrated that an important element in the suitability of the layer on which the nanotube grow is the smoothness of the surface, i.e., experiments conducted by the inventors herein have shown a strong correlation of nanotube growth success with surface smoothness, for a range of substrate surface textures and materials (such as thermally grown SiO2, CVD formed SiO2, Si3N4, and amorphous SiO2). It was demonstrated (e.g., by atomic force microscopy or “AFM”) that the longest nanotubes were formed under given CVD conditions on the smoothest surface, and that the most truncated nanotubes being grown on the roughest surfaces. It is believed that the nanotube growth includes the tubes being “spun” out from metal particles supersaturated with carbon. The decreased length of nanotube are generally grown on rough surfaces, as the higher incidence of surface defects and features tend to interact with growing nanotubes, so as to stop the nanotube growth process.

In one embodiment, a NT growth favoring layer comprises a layer of SiO2, which is preferably from about 50 to about 2000 nm thick, and more preferably from about 50 to about 1000 nm thick (which may be formed by methods such as CVD, EPCVD, and the like).

Note that the optimum dimensions and properties of the contaminant protective layer and the surface NT growth favoring layer depend on the particular device application architecture and operating conditions. One of ordinary skill in the art applying the inventive methods and devices can readily adjust the thickness, composition and other properties of either or both of the contaminant protective layer and the surface NT growth favoring layer, so that for a particular nanotube field effect transistor embodiment, the gate voltage, the gate leakage current, the on-off current ratio, and/or other operational characteristics of the completed device are within selected limits.

Alternatively, the diffusion barrier element (contaminant protective layer) and the surface NT growth favoring layer element may be combined into an integrated layer. For example, a the integrated surface layer may comprise a material which has a high dielectric properties, high metal diffusion resistance and a very smooth surface texture.

In certain embodiments, a film is formed upon the bulk semiconductor substrate which has a selected dielectric constant and high metal diffusion resistance (e.g. Si3N4, and the like) The deposited film is then modified to a desired level of smoothness (e.g., by CMP, thermal annealing, and the like. Alternatively, the film can be deposited by a method producing a smooth surface as-deposited (e.g., using a high density plasma deposition system where sputtering and deposition occur simultaneously).

Pre-Patterned Substrates. A wafer embodiment having aspects of the invention comprises a pre-patterned semiconductor substrate wafer (e.g., silicon), preferably of generally conventional semiconductor wafer size, shape, thickness and other characteristics, so as to be suitable for processing, treating, cleaning, masking, testing and handling by conventional semiconductor fabrication equipment. The wafer includes at least one contaminant-protective layer or diffusion barrier (such as described herein) upon the bulk substrate on at least an upper side of the wafer. Preferably, the wafer further comprises a NT growth favoring layer (such as described herein) overlaying the contaminant-protective layer. The wafer embodiment is suitable to be provided to users for the making of sensors and/or other NT electronic devices.

Optionally, the wafer may include a pre-deposited dispersal of a NT growth promoter composition (such as described herein) disposed upon the substrate (e.g., upon a NT growth favoring layer). In certain embodiments, the growth promoter may be arranged at selected sites or pattern in which NT growth is desired, and excluded from portions in which NT growth is not desired.

Optionally, the wafer may include a pre-deposited pattern of conductors or electrodes (or other electronic devices or elements) upon or adjacent at least the upper surface. The electrode pattern may include electrodes for a plurality of sensors, for example disposed in an array. Notably, wafer-level fabrication may be advantageous for manufacturing both single sensors and integrated arrays of sensors.

A method embodiment having aspects of the invention comprises, in any operative order, the steps of:
a) providing a pre-fabricated semiconductor wafer (e.g., silicon);
b) forming at least one contaminant-protective layer (such as described herein) upon the bulk substrate on at least an upper side of the wafer;
c) forming a conditioned surface layer (such as described herein) disposed on at least an upper side of the wafer;
d) optionally forming a pattern or dispersal of a NT growth promoter composition (such as described herein) disposed upon or adjacent at least an upper side of the wafer; and
e) optionally forming a pattern of conductors, electrodes, and/or other device elements disposed upon or adjacent at least an upper side of the wafer.

### 4. EXAMPLE A

### Hydrogen Functionalization of a Nanotube Network Sensor

A number of sensing devices 10 generally similar to that of FIG. 1 were made as described above. The devices included a back-gate 4, controllably biased to permit operation as a nanotube-channel field effect transistor (NTFET). Initial testing of samples was performed without the deposition of functionalization material. Comparison testing of samples was performed following deposition of various functionalization materials, and upon exposure to a variety of environments, as described below. The device has substantially only semiconducting nanotubes included in the nanotube layer 2.

In the measurement methodology, the devices were contacted with pin probes either in air or in a sealed chamber. In certain measurements devices were heated in the chamber in vacuum of 10-6 torr to temperatures of 200° C. For in-air testing, a hypodermic tube positioned over the chips was used to introduce gas. Hydrogen (ultrapure electronic grade, Airgas) was diluted with argon (Airgas) from 100% to 20 ppm. Hydrogen concentrations in the data presented herein refer to these mixtures. Gas flow was controlled by a regulator followed by a toggle valve. Most measurements involved only brief gas exposures. For vacuum testing, the chamber with devices in it was evacuated for an hour between measurements. Hydrogen gas was introduced up to various static pressures. These pressures are reported in the data for vacuum testing.

Effect of palladium (Pd) coating on NTFET. FIG. 4A shows the characteristics of a “bare” nanotube FET device in air without hydrogen. A typical transconductance versus gate voltage of an NTFET device without the deposition of functionalization material is displayed, showing a generally smooth decrease in conductances with increasing gate voltage, from an “on” value at Vg of −10 volts to an “off” value at about +7 volts. In contrast, FIG. 4B shows the characteristics of a NTFET devices with palladium (Pd) coating. After deposition of a coating 7 comprising palladium over the NT network layer 2, the conductances of NTFET devices were found to be insensitive to gate voltage, as indicated in top part of FIG. 4B. The conductances of devices with Pd coating corresponded neither to the “on” nor the “off” values of the undecorated devices.

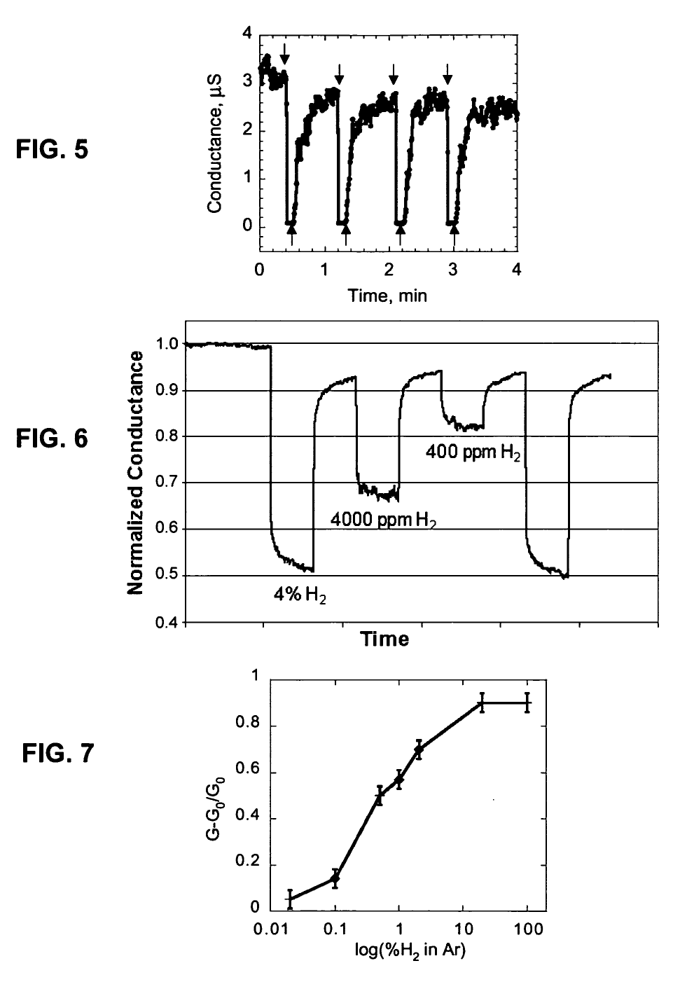

Exposure to hydrogen In air. An NTFET device 10 with palladium coating 7 was exposed to pure hydrogen to characterize their saturated response. As shown in FIG. 4B, typically the NTFET exhibited a significant decrease in conductance to values equal to the “off” conductance of the undecorated devices, measured at −10V gate voltage. FIG. 5 illustrates the response of a sensor to short bursts of pure hydrogen in a surrounding air environment. Disruption of the hydrogen flow, and the consequent removal of the hydrogen environment led to the recovery of the devices to conductance values observed before the application of hydrogen.

As shown in FIGS. 6 and 7, the NTFET device conductances decreased smoothly to a steady value which varied with hydrogen concentration. The difference between the steady response conductance and the initial “on” conductance for a typical device is plotted in FIG. 7 with respect to hydrogen concentration.

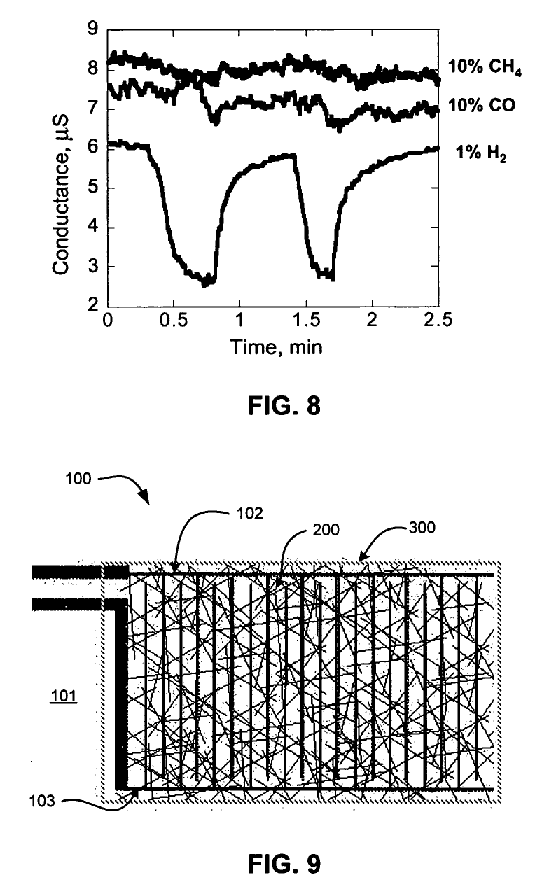

Low Cross-Sensitivity. Embodiments of NTFET sensors having aspects of the invention readily respond to hydrogen at room temperature. Operation at room temperature conveys numerous advantages, particularly with respect to specificity and poisoning. FIG. 8 shows the response of palladium-coated devices to carbon monoxide and methane. These common gases react readily on hot catalysts. In traditional sensors the presence of either produces a false positive signal. At room temperature, they react slowly or not at all. The palladium-coated NTFET devices respond to them about 1000 times more weakly than to hydrogen. It should be understood that the functionalization material and composition may be optimized to minimize cross-sensitivity to a particular non-target species.

### 5. EXAMPLE B

### Nanotube Network H2 Sensor with Remote Measurement Circuitry

An exemplary NTFET sensor with measurement circuitry having aspects of the invention may be made as follows. A degenerately doped silicon wafer with a silicon oxide film may be coated with carbon nanotubes in a random network, as described in the above incorporated U.S. patent application Ser. No. 10/177,929. As shown schematically in FIG. 9, titanium contacts 35 nm thick covered with gold contacts 100 nm thick were deposited and patterned by photolithography and lift-off to form opposing contacts 100, 110. Contacts 100, 110 each comprised a plurality of interdigitated portions disposed over a generally rectangular region 300. A network of randomly oriented nanotubes 200 is disposed over the silicon substrate. Nanotubes in the network 200 are in electrical contact with interdigitated portions of contacts 100, 110.

After the deposition of the contacts 102, nanotubes outside of the generally rectangular area were removed by oxygen plasma etching, leaving nanotube network 200 remaining. The use of interdigitated sets of metal electrodes 100, 110 with nanotube network 200 interposed generally between the interdigitated contacts results in many nanotubes connected in parallel across the electrodes 100, 110. When the conductance between two sets of wires on a die was measured as a function of gate voltages between +10 V and −10 V, the maximum conductance was approximately 10 kΩ, and the on-off ratio was approximately 10.

To functionalize for hydrogen, palladium metal was deposited on the substrate and patterned using photolithography and lift-off. To facilitate economic production, the above described process may be carried out on a plurality of sensors in parallel fashion, such that multiple copies of the sensor geometry are arranged over the wafer, each copy being made as an individual sensor die that can be separated from the wafer or diced for further packaging into a completed measurement apparatus.

A selected die was separated from the wafer. The die may be mounted in a standard chip package (e.g. a standard 16-pin chip carrier) for subsequent incorporation into measurement circuitry, or alternatively the die may be mounted directly onto a circuit board. For example, the sensor die may be mounted directly on a printed circuit board, with wires connecting the interdigitated wires on the chip to contacts on the printed circuit board.

In this example, several calibration values were measured, at which the electrical resistance of the sensor was recorded in the presence of a known concentration of hydrogen in air.

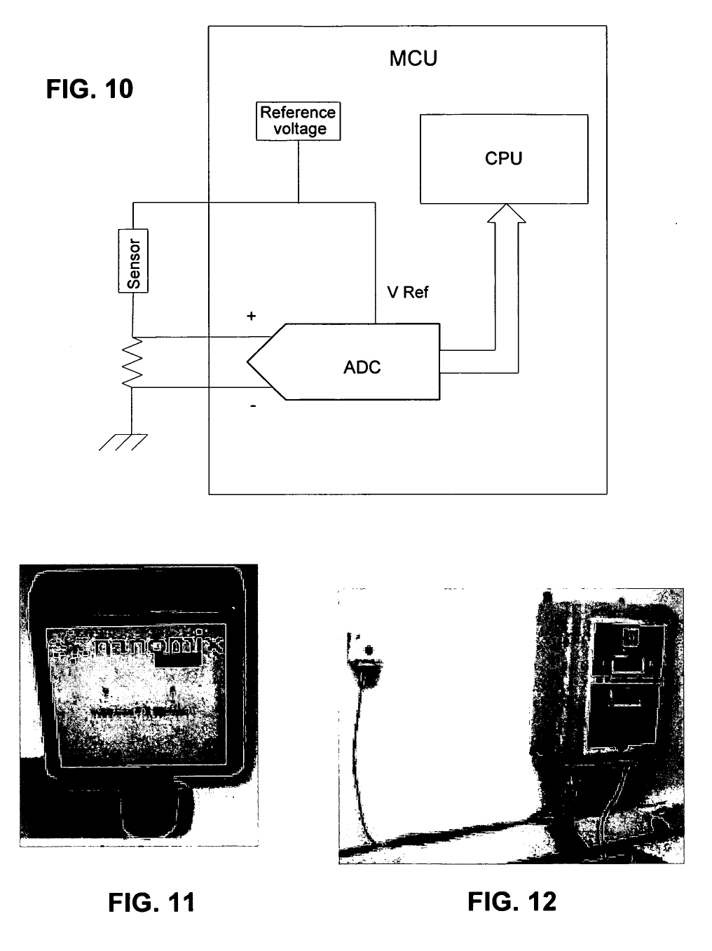

An electronic circuit was built as shown in FIG. 10, using integrated circuit chips mounted on the same circuit board as the sensor die. A microprocessor was selected (Texas Instruments part MSP430F1232) which includes an integrated 10-bit analog-to-digital converter and a voltage source. The voltage source applied a bias voltage across the nanostructure sensor in series with a resistor. The analog-to-digital converter measured the voltage across the resistor and converted this voltage into a digital signal. The microprocessor recorded the digital signal. Given the known value of the resistor and the digital signal recording the voltage across the resistor, the microprocessor calculated the current through the resistor. Given the known bias voltage and the digital signal recording the voltage across the resistor, the microprocessor calculated the voltage across the nanostructure sensor. Given the voltage across the nanostructure sensor and the current through the resistor, the microprocessor calculated the resistance of the nanostructure sensor. The microprocessor had previously been loaded with the measured calibration value of the nanostructure sensor. It used these values to convert the measured resistance of the sensor into a concentration of hydrogen. A photograph depicting the remote sensor package is included in FIG. 11.

The microprocessor communicated data to a radio communications control chip (Chipcon part CC2420). The radio communications control chip transmitted the carbon dioxide concentration at a radio frequency of 2.4 GHz, using the 802.15.4 standard for radio communication. A base station (Chipcon part CC2420 integrated with a computer) received the data communicating the carbon dioxide concentration. A battery supplied power to the remotely communicating device. A photograph depicting the base receiver station is included in FIG. 12.

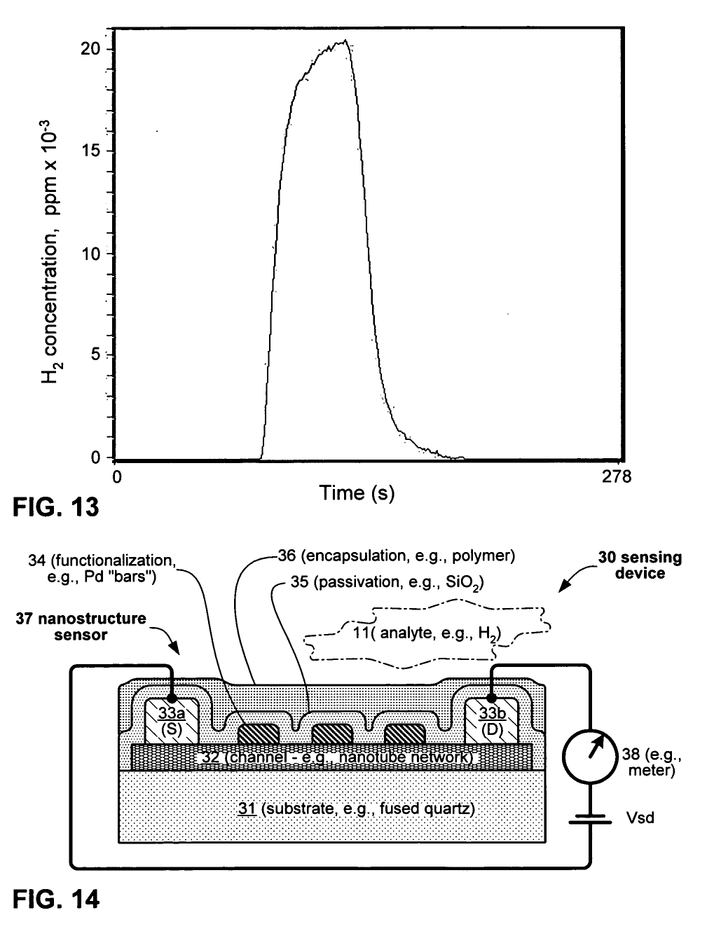

FIG. 13 is a plot showing a measurement by an exemplary sensor system embodiment having aspects of the invention, generally similar to that of FIGS. 9-12. The plot shows calibrated values of H2 as parts-per-million (ppm) in air versus time. It can be seen that upon exposure the H2 (in this case at a concentration of about 20,000 ppm or about 2%), the sensor rapidly responds to an asymptotic output value. As hydrogen exposure is removed, the sensor rapidly recovers to its pre-exposure reading.

Temperature and Pressure Compensation. Optionally, measurement precision and accuracy may be enhanced by use of compensation methods, for example, multiple linear regression (MLR) for temperature and humidity. A similar approach will be used with pressure instead of relative humidity. The compensation method may include electrical measurements of the sensor, such as resistance, polynomial terms to account for nonlinearity, temperature and pressure measurements, and interactions. For example, a product of temperature and resistance can be used to account for change of sensitivity with temperature:
C=k₀+k₁R+k₂T+k₃RT+ε
where C is gas concentration, R is sensor resistance, T, temperature and ε, regression error.

The calibration data are typically generated using the classic full factorial experiment design. The factors are the analyte gas concentration, temperature, pressure and even relative humidity. The levels may be balanced in such a way that any sensor drift will not be confused with response to such factors or interactions of factors. Collected data may be stored in a relational database, and MLR performed. The resulting coefficients and goodness-of-fit statistics may also be stored in a database. These coefficients can be applied to data from other tests for validation, and/or programmed into the sensor system firmware. Principal component analysis (PCA) may be performed on test data, to makes it possible to represent multivariate data in fewer dimensions. Temperature sensors, such as a thermistor may be included in the sensor system to permit automatic electronic compensation for the variation of temperature.

### 6. EXAMPLE C

### Passivated/Encapsulated H2 Sensor

FIG. 14 shows schematically in cross section an exemplary sensor device 30 having aspects of the invention made using the methods described above. The sensing device 30 includes a nanostructure sensor 37 which makes use of an insulating substrate 31 which may comprise a dielectric material, such as fused quartz and the like.

The employment of a dielectric substrate material provides electrical isolation of the channel 32 and source and drain contacts 33a, 33b. Optionally, a separate dielectric layer or other electrical isolation element may be included (not shown). In the embodiment of Example A, the channel 32 comprises a carbon nanotube network grown by CVD directly upon the substrate 31 by the methods described above. Optionally, a conditioned surface layer can be included to favor nanotube growth. Alternatively, the channel 32 may be formed of alternative materials and methods as described above.

In the embodiment of Example A, an optional passivation layer 35 (e.g., comprising SiO2) and an optional encapsulation material 36 (e.g., comprising a polymer coating) are included. The composition and/or configuration of the passivation and/or encapsulation layers may be optimized to permit diffusion of a target analyte 11 (in Example A this includes gaseous hydrogen in air) to interact with functionalization 34 adjacent nanostructured channel 32). Likewise, these layers may be optimized to exclude or reduce diffusion of non-target species. It has been found that passivation and encapsulation layers may be useful in reducing cross-sensitivity and/or humidity sensitivity.

The sensor device 30 includes suitable circuitry and instrumentation to perform measurements of the electrical response of nanostructured sensor 32 to analyte 11, represented schematically as a source drain voltage source Vsd, and measurement instrumentation, represented by meter 38. Note that no gate electrode is shown in FIG. 14, although a gate electrode may optionally be included, including corresponding circuitry (optionally including additional dielectric as required for isolation from channel).

Encapsulation. As shown in FIG. 14, an optional encapsulation layer 36 may be included, covering all or a portion of the device. Encapsulation layer 66 may comprise an insulator material such as SiO₂, SiO, Si₃N₄, Al2O3, or other organic layers such as Parylene, organic polymer, hydrophobic polymer (fluoropolymers for example), parylene, or a multiplayer of such materials. The encapsulation layer 36 may be selected so as to reduce cross sensitivity and/or to act as a water barrier in order to reduce water induced response variations, and/or to protect the device from mechanical damage. In certain embodiments, the encapsulation layer 36 is a multiple layer or composite structure. It has been shown that multiplayers of differing materials such as Al2O3/polymer can reduce the diffusion of water by multiple orders of magnitude. Such a structure may be more efficient than a thicker layer of one material alone, because it reduces the possible number of pathways for the water molecules (such as pinholes, cracks, and the like).

In alternative exemplary embodiments of an H₂ sensor having aspects of the invention, it has been shown that without the encapsulation layer 66 (See FIG. 1), the response of the device may be dependant on the relative humidity of the sample medium, e.g. ambient air. The encapsulation layer 36 allows reducing the variation induced by the relative humidity.

In addition an encapsulation layer 36 may increase the specificity of the sensor to hydrogen in contrast to other analytes. Since hydrogen is highly diffusive, H₂ may pass easily through an encapsulation layer which is generally impermeable to other (typically larger) molecules that may be present in the sample medium. For example, it may be found that without the encapsulation a large variation of the conductance of the sensor can be measured if it is exposed to some gas such as NH3 or NO2, and that an encapsulation layer may be readily configured to eliminate or substantially reduce this cross sensitivity.

A preferred encapsulation layer 36 comprises about 5 nm thickness of SiO₂ and about 1 to about 3 μm of AFP4. An alternative encapsulation layer may comprise about 5 to about 15 nm of Al2O3 and about 1 to 3 μm of AFP4. The addition of such an encapsulation layer does not greatly reduce the sensor sensitivity or response time to hydrogen. The encapsulation layer may also increase the useful lifetime of the sensor.

Polymers have the additional advantage of being readily processable using procedures such as spin coating, dip coating, drop casting, and microspotting. Microspotting, in particular, may be useful for fabrication of multiple sensor in a sensor array that is configured to respond to a variety of different analytes. Yet another advantage is that polymer coatings often modify the characteristics of NTFET devices, which can be monitored during processing for control of coating processes.

Passivation. In addition or alternatively to an encapsulation layer, the device may include passivation of the contacts and/or other device elements. As shown in FIG. 10, an optional passivation layer 35 may be included, covering all or a portion of the contacts 3, and/or the nanotube layer 2, and/or any exposed substrate 1, or all of these. The passivation layer 35 can comprise an inorganic insulator material such as SiO₂, SiO, Si₃N₄, Al2O3 and the like. Alternatively or additionally, the passivation layer 35 may comprise organic layers such as parylene, organic polymer, hydrophobic polymer (fluoropolymers for example), or any multilayers of such materials.

As an unanticipated result, it is shown that a combination of passivation and encapsulation may be selected to provide a device structure that gives good carbon nanotube stability over long useful sensor life, and at the same time stabilizing the chemical reaction on the metal layer with the polymer to make it relative humidity independent, and providing a very useful sensitivity and selectivity over a wide range of analyte concentrations. Further description of passivation of all or a portion of an NT electronic device is contained in U.S. patent application Ser. No. 10/280,265 filed Oct. 26, 2002 entitled “Sensitivity Control For Nanotube Sensors” (published as US 2004-0043527), which is incorporated by reference.

### CONCLUSION

Having thus described a preferred embodiment of the nanotube sensor device, it should be apparent to those skilled in the art that certain advantages of the within system have been achieved. It should also be appreciated that various modifications, adaptations, and alternative embodiments thereof may be made within the scope and spirit of the present invention.

1. A nanostructure sensing device, comprising:
a substrate;
a first nanostructure disposed over the substrate, the nanostructure having a functionalization sensitive to at least one selected analyte;
a first conductive element disposed over the substrate and forming a first electrical junction with a first nanostructure; and
a second conductive element disposed over the substrate and forming a second electrical junction with the first nanostructure.

2. The nanostructure sensing device of claim 1, wherein the first nanostructure includes one or more nanoparticles selected from the group consisting of nanotubes, nanowires, nanofibers, and nanorods.

3. The nanostructure sensing device of claim 1, further comprising a gate electrode in proximity to the first nanostructure.

4. The nanostructure sensing device of claim 3, wherein the gate electrode includes at least one of an undifferentiated gate electrode, a back gate electrode, a top gate electrode, a liquid gate electrode, and a side gate electrode.

5. The nanostructure sensing device of claim 2, wherein the first nanostructure comprises a network of nanotubes.

6. The nanostructure sensing device of claim 5, wherein the first nanostructure comprises a network including a plurality of interconnecting single wall carbon nanotubes.

7. The nanostructure sensing device of claim 5, wherein the substrate comprises a surface layer structure facing the first nanostructure.

8. The nanostructure sensing device of claim 7, wherein surface layer structure comprises a material selected from the group consisting of semiconductors, semiconductor oxides, semiconductor nitrides, and combinations thereof.

9. The nanostructure sensing device of claim 7, wherein the surface layer structure comprises a top layer configured to favor the catalytic growth of carbon nanotubes.

10. The nanostructure sensing device of claim 9, wherein the top layer top layer comprises a nano-scale smooth layer of SiO2.

11. The nanostructure sensing device of claim 7, wherein the surface layer structure comprises a substrate contaminant protection layer.

12. The nanostructure sensing device of claim 11, wherein the substrate contaminant protection layer comprises a diffusion barrier to metals.

13. The nanostructure sensing device of claim 12, wherein the diffusion barrier to metals comprises Si3N4.

14. The nanostructure sensing device of claim 12, wherein the diffusion barrier to metals comprises a layer of Si3N4 from about 50 to about 3000 nm thick.

15. The nanostructure sensing device of claim 12, wherein the diffusion barrier to metals comprises a layer of Si3N4 from about 50 to about 1000 nm thick.

16. The nanostructure sensing device of claim 7, wherein surface layer structure comprises a gate dielectric.

17. The nanostructure sensing device of claim 1, wherein the functionalization is sensitive to hydrogen.

18. The nanostructure sensing device of claim 17, wherein the functionalization comprises at least a partial coating of the nanostructure with at least one transition metal

19. The nanostructure sensing device of claim 18, wherein the transition metal is selected from the group consisting of palladium, platinum, and nickel.

20. The nanostructure sensing device of claim 18, wherein the at least one transition metal comprises Pd in an alloy including one or more elements selected from Pt, Au, Ni, Ti, and Cu.

21. The nanostructure sensing device of claim 20, wherein the at least one transition metal comprises Pd in an alloy including about 16% w/w of Ni.

22. The nanostructure sensing device of claim 5, wherein the network is formed on the substrate using a chemical vapor deposition process.

23. The nanostructure sensing device of claim 5, wherein the network is formed by means of vacuum deposition of a nanotube fluid suspension upon a porous surface.

24. The nanostructure sensing device of claim 1, further comprising passivation material covering at least a portion of the first nanostructure.

25. The nanostructure sensing device of claim 24, wherein the passivation material substantially covers the portions of the nanostructure which are contiguous with the first electrode and the second electrode.

26. The nanostructure sensing device of claim 24, wherein the passivation material is impermeable to at least one species.

27. The nanostructure sensing device of claim 24, wherein the passivation material is substantially impermeable to water.

28. The nanostructure sensing device of claim 24, wherein the passivation material comprises more than one layer of material.

29. The nanostructure sensing device of claim 24, wherein the thickness of the passivation material is chosen to tune sensitivity to the analyte of the nanostructure sensing device.

30. The nanostructure sensing device of claim 24, wherein the passivation material includes at least one constituent selected from the group consisting of silicon oxides, fluorinated, hydrogenated and carbonated silicon oxides, silicon nitride, metal oxides, and combinations thereof.

31. The nanostructure sensing device of claim 24, wherein the passivation material includes at least one constituent selected from the group consisting of Teflon™, Nafion™, polyethylene and polypropylene.

32. The nanostructure sensing device of claim 24, wherein the passivation material includes at least one constituent selected from the group consisting of SiO2, SiO, Si3N4, A12O3, Parylene, organic polymer, hydrophobic polymer, and a fluoropolymer.

33. The nanostructure sensing device of claim 24, wherein the passivation material reduces cross sensitivity of the device.

34. The nanostructure sensing device of claim 24, wherein the passivation material is a least partially permeable to hydrogen.

35. The nanostructure sensing device of claim 34, wherein the thickness of the passivation material is chosen to tune selectivity for hydrogen of the nanostructure sensing device.

36. The nanostructure sensing device of claim 35, wherein the passivation material has a thickness between about 3 nm and 500 nm.

37. The nanostructure sensing device of claim 1, wherein the functionalization sensitive to at least one selected analyte includes at least one recognition material, wherein the recognition material is disposed adjacent the nanostructure, and wherein the recognition material is separated from the nanostructure by a dielectric layer.

38. The nanostructure sensing device of claim 37, wherein the nanostructure includes a network of carbon nanotubes disposed upon a substrate.

39. The nanostructure sensing device of claim 38, wherein at least a portion of the recognition material is conductive, and wherein at least a portion of the recognition material is connectable to an electrical lead so as to form a gate electrode.

40. A substrate for a nanoelectronic device, comprising:
a substrate base;
a first layer disposed over the substrate base comprising a material substantially impermeable to the diffusion of metallic elements during carbon nanotube growth upon the substrate by chemical vapor deposition; and
a second layer disposed over the first layer and comprising a material configured to favor the catalytic growth of carbon nanotubes.

41. A substrate as in claim 40, wherein the substrate base comprises a semiconductor material.

42. A substrate as in claim 41, wherein the semiconductor material comprises silicon.

43. A substrate as in claim 40, wherein the first layer comprises Si₃N₄.

44. A substrate as in claim 43, wherein the first layer is from about 50 to about 3000 nm thick.

45. A substrate as in claim 43, wherein the first layer is from about 50 to about 1000 nm thick.

46. A substrate as in claim 40, wherein the second layer provides a surface promoting consistent growth of carbon nanotubes upon the substrate by chemical vapor deposition.

47. A substrate as in claim 40, wherein the second layer comprises SiO₂.

48. A substrate as in claim 40, wherein one or more of the first layer and the second layer comprises a gate dielectric.
